(19) United States
(12) Patent Application Publication
(10) Pub. No.: US 2005/0037503 A1
(43) Pub. Date: Feb. 17, 2005

(54) Methods for preventing or reducing sources of halogenated volatile organic compound impact to groundwater

(76) Inventor: James L. Peterson, Princeton, NJ (US)

Correspondence Address: McDERMOTT, WILL & EMERY, 600 13th Street, N.W., Washington, DC, 20005-3096, US

(21) Appl. No.: 10/639,528

(22) Filed: Aug. 13, 2003

(51) Int. Cl. G01N 33/00

(52) U.S. Cl. 436/55

(57) ABSTRACT

Methods for preventing or reducing halogenated volatile organic compounds in soil are disclosed. The methods include analyzing soil near areas that have been exposed to deicing activity, such as the application of sodium chloride salt to melt ice and snow. The soil is analyzed to determine one or more threshold condition for halide-salt enhanced halogenation of organic matter in the soil. Remedies include reducing the severity of the threshold condition or conditions that lead to halogenation of organic compounds in the soil. These remedies include reducing or eliminating halide-salt influxes through use of an alternate, non-halide deicer and modifying soil characteristics conducive to HVOC formation by the addition of lime and/or fertilizer.

### FIELD OF THE INVENTION

The present invention relates to methods for preventing or reducing halogenated volatile organic compounds in soil. In particular, the present invention relates to the prevention and treatment of soils that are exposed to salt deicing activity which can cause the formation of halogenated organic compounds in the soil environment which in turn can contaminate groundwater.

### BACKGROUND

Various studies have shown that halides entering soil with precipitation can cause the natural formation of organohalogens, including chloroform and other halogenated volatile organic compounds (HVOCs), and that natural transport mechanisms may cause their downward migration within the soil profile, resulting in groundwater contamination. This natural, in-situ formation of organohalogens takes place via the interaction of halides and organic matter, assisted by enzyme-mediated and other biological processes and/or through purely chemical interaction with ferric iron. (Gribble “Naturally Occurring Organohalogen Compounds” Acc. Chem Res. 1998:31:141-152)

Hoekstra, et al. (Hoekstra et al. “Natural Formation of Chloroform and Brominated Trihalomethanes in Soil” Envirn. Sci. Technol. 1998:32:3724-3729) describes the natural formation of chloroform in soil by a process involving the formation of reactive chlorine species, such as hypochlorous acid, from chloride and hydrogen peroxide by a chloroperoxidase (CPO)-mediated reaction followed by reaction of the chlorine species with organic matter, such as humic matter, equations 1 and 2 respectively.
H₂O₂+H⁺+Cl⁻→CPO→HOCl+H₂O [eq 1]
Humic material+HOCl→chlorinated humic material+CHCl₃+CCl₃COOH+ [eq 2]
If bromine is present, Hoekstra et al. report that brominated compounds can also form by a similar pathway.

Specific chlorinated volatile organic compounds (VOCs) shown to form via these processes to date include chloroform (a.k.a., chloromethane), methyl chloride and dichloromethane. In-situ halogenation processes have also been shown to produce brominated and iodinated VOCs. Factors associated with increased organohalogen formation included increased soil organic matter content, increased iron content, acidic (low pH) soil conditions and increased halide input. In-situ organohalogen formation appears also to depend on soil moisture content; the process takes place more readily in moist soils than in dry soils.

The United States Environmental Protection Agency (USEPA) has identified chloroform as a potential cause of liver, kidney or central nervous system problems and of increased risk of cancer. The United States Primary Drinking Water Regulations currently specify a Maximum Contaminant Level (MCL) of 80 ug/L for total trihalomethanes (THMs), which include chloroform, bromoform, bromodichloromethane and chlorodibromomethane. Individual states have set even lower numeric standards specifically for chloroform (e.g., New Jersey Class II-A Groundwater Quality Standard of 6 ug/L).

Chloroform is one of the most commonly detected VOCs in surveys of raw, untreated groundwater supplies. Its presence in groundwater is often attributed to leakage of sanitary sewers and chlorinated water supply piping, as chloroform and other trihalomethanes are known by-products of chlorine disinfection of wastewater and drinking water. Localized chloroform detections in untreated groundwater are sometimes also attributed to shock chlorination treatment to remove biological growths in wells. It is unlikely that these proposed sources account for all of the chloroform detections and other sources likely play a significant role, however.

HVOC contamination of groundwater along salt-treated roadways has not been extensively documented. Studies of such areas typically include analysis of samples for salt constituents (e.g., sodium, calcium and chloride) but not for HVOCs.

Because concentrations of chloroform (and possibly other HVOCs) formed via salt-enhanced in-situ halogenation of organic matter can exceed health-based drinking water and groundwater remediation standards, a process is needed to prevent the in-situ formation of halogenated volatile organic compounds as described which leads to their accumulation in drinking water, and to remedy existing sources of such contamination. Specific challenges to be met in mitigating such sources include identification of possible areas of impact and implementation of a remedy which minimizes HVOC formation while offering continued deicing and maintenance of public safety.

### SUMMARY OF THE INVENTION

Advantages of the present invention include preventing and reducing HVOC in soil and groundwater.

These and other advantages are satisfied, at least in part, by methods of preventing or reducing at least one halogenated volatile organic compound (HVOC) from forming in the soil environment. The method comprises assessing characteristics of soil that is exposed to deicing activity to locate possible areas of concern and/or analyzing groundwater to identify actual areas of HVOC impact. Deicing activity includes the application of salt, such as sodium chloride, to an area, such as roads, parking lots, walkways, etc. to melt or prevent the formation of ice and snow. During the application of salt, it has been noted that the salt can contact surrounding soil and when there is a threshold condition, enhance the formation of halogenated matter, including organohalide compounds, in the soil. These organohalogen compounds include volatile organic compounds that adversely effect the soil and can contaminate groundwater. The method identifies these threshold conditions and reduces the severity of threshold conditions to prevent the formation of the HVOC in the soil.

Embodiments of the present invention include analyzing soil for a threshold condition including (1) organic matter content, (2) soil having a high halide influx from deicing activity, (3) soil having a low pH, i.e., soil having a pH of about 4.5 or less, (4) soil having moderate water content. Additional embodiments include reducing the severity of any one of these threshold conditions by increasing the pH of the soil and/or reducing the influx of halide salts at or near the soil, as by applying a substitute non-halide salt, such as a calcium magnesium based salt.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention.

### DETAILED DESCRIPTION OF THE DISCLOSURE

It was discovered, through analysis and investigation, that the formation of volatile organohalides in soil can be enhanced by an influx of halide-salts, such as those typically used for deicing paved surfaces. It was further discovered that the enhanced production of HVOC in soil results in high levels of HVOC contaminated groundwater.

As used herein, soil includes the land surface and the entire subterranean environment thereunder. The subterranean environment includes the soil solid phase, pore water and soil gas phase. Typically, the soil most affected by halide-salts is the surface and about five to fifteen feet immediately below the surface and typically above the water table. While not intending to be bound by any theory, it is believed that halides from deicing activity interact with carbonaceous components and form HVOCs in this environment. For example, chloroform can form in any phase or level of the soil depending upon certain conditions. Such chlorinated volatile compounds resulting from the use of sodium chloride is particularly problematic, but all HVOCs are included in the present invention. The present invention contemplates the reduction and/or elimination of the source of HVOC formation and/or the HVOC itself wherever it occurs or is located in or on the soil. This in turn reduces the amount of HVOC impacting groundwater.

In one investigation, it was determined that a plume of impacted groundwater exhibited a significantly high chloroform concentration, up to 110 micrograms per liter (ug/L). (Chloroform concentrations in groundwater of up to 1.6 ug/L have been reported in a natural setting away from anthropogenic sources of chloride influx.) An extensive sampling and analysis program revealed that the plume originated near a parking lot and concrete walkway that had been treated with salt, i.e., sodium chloride, for snow and ice removal since the mid-1980s. Runoff of deicing meltwater to adjacent organic topsoil took place in this area. Acidic soil conditions were also documented. After a careful and detailed investigation ruled out the possibility of other contributing sources, it was concluded that natural in-situ halogenation of soil organic matter, augmented by influxes of chloride-containing runoff associated with the deicing salt applications, caused enhanced formation of chloroform in the soil, leading to the observed groundwater contamination.

Conditions similar to those observed at this site exist at other locations where halide-salts, e.g., sodium chloride, have been used for deicing. Salt application is the most frequently used method of roadway deicing and over 15 million tons of salt are applied to roads in the United States annually for this purpose. Annual salt application rates on highways range from 300 to 800 tons per two-lane mile. Mainly as a result of plowing, splashing by cars and runoff, much of the salt applied to the road finds its way to the soil of the roadside environment. There, it can come into contact with organic-rich soils (e.g., natural topsoils and vegetative organic soils of landscaped medians and highway margins). In addition, acidic soils and soils with elevated iron content are common, so conditions that may favor salt-enhanced formation of HVOCs appear to be widespread.

Throughout this document, the term “deicing” is meant to refer to the processes undertaken and materials used to prevent the accumulation of and facilitate the removal of, snow and ice from surfaces. The term “deicing area” refers to the areas where deicing takes place, but also includes areas such as salt stockpiles from which halide-containing chemicals/products used for deicing may migrate and cause environmental impairment. The term “halide-salt” refers to halide-containing deicing chemicals, including sodium chloride, calcium chloride, magnesium chloride and potassium chloride, and to mixtures containing these compounds. Other halide-based salts, such as bromine salts, iodine salts, etc., are also contemplated within the meaning of halide-salt.

HVOC formation in soil can be prevented or minimized by first identifying potential threshold conditions or values which promote its formation and then implementing a procedure to prevent or remedy the formation of HVOC in soil. Identifying threshold conditions includes an analysis to evaluate co-occurrence of factors associated with salt-enhanced in-situ halogenation of organic matter and can include a field investigation to evaluate actual halogenated volatile organic compound contamination. One remedy to prevent the formation of HVOC is based on discontinuance of halide-salt use with substitute application of an alternative, non-halide containing deicing chemical with properties conducive to discontinuing the interaction between halide, i.e., chloride, and organic matter. This approach advantageously outlines a means of identifying previously undetected environmental impacts from road salting and implements a process for preventing or discontinuing deicing-related chloride influx (preventing new HVOC activity) while maintaining an effective deicing program and rendering residual halide from past salt applications less likely to function as an ongoing source of HVOC activity.

In one aspect of practicing the present invention, a two-step process is implemented for identifying potential impacts and sources of salt-enhanced in-situ halogenation of organic matter. This includes: (1) a screening analysis to identify areas with increased likelihood of HVOC formation resulting from salt applications; and (2) a field investigation to evaluate actual HVOC formation which can result in groundwater impact.

In certain instances, a general area of impact may have been previously identified (e.g., existing monitoring data indicating chloroform groundwater impact near a roadway with no other evident sources) for the chloroform. Under such circumstances, the need to perform screening activities may be limited and assessment may proceed directly to delineation activities. Similarly, the screening activities might be employed in certain situations where follow-up with field investigation is not currently possible. Such an approach might be used by government agencies to provide an initial assessment of potential impact along extensive road networks where salt has or may be applied for deicing. Detailed descriptions of Steps 1 and 2 are provided below.

Step 1: Screening Analysis

The screening analysis includes identifying areas where certain threshold conditions associated with salt-enhanced in-situ halogenation of organic matter occur together. Such conditions include road salt application, areas of sufficient organic matter content, high iron content, e.g., high ferric iron content, low soil pH (acidic soils) and moist soil conditions.

In some instances, this information is already available for a given target area. Published information regarding salt application to roadways is generally available from government agencies responsible for maintenance of public roadways, while obtaining such information for privately owned land requires inquiries with the landowner. Soil survey reports such as those prepared by the United States Department of Agriculture (USDA) provide detailed mapping of the locations of soil units exhibiting specific physical and chemical properties. Typically, these reports include qualitative information and/or chemical analytical data for a number of soil analytical parameters, including pH, organic carbon, iron and moisture content. Similar information is frequently included in maps and reports prepared by geological surveys, such as the United States Geological Survey or a State's geological information, e.g., the New Jersey Geological Survey.

In reviewing the data for each of these conditions, screening thresholds should be utilized to identify conditions which may contribute to salt-enhanced in-situ halogenation of organic matter. Examples of determining a threshold condition for halide-salt enhanced halogenation of organic matter in soil is provided below.

Salt Application—The nature of chemical deicing is such that much of the salt applied for this purpose finds its way to the soils adjacent to the salt treated pavement. Only in limited areas (e.g., where roadway margins are paved or where engineered drainage structures effectively prevent it) will adjacent soil remain free of such impact. Therefore, subject to field determination of actual conditions, use of deicing halide-salt in a given area may be taken as a likely indication of halide impact to adjacent soils, which may enhance in-situ halogenation of organic matter. Attention should also be paid to areas of road salt storage and snow and ice dumping, as these represent possibly large sources of salt runoff and accumulation, respectively.

Organic Carbon Content of Soil—The organic carbon content of soil at a given location is considered to exceed its threshold condition when any of the following conditions are present: (1) Soil units consisting of peat, muck or other soil defined as “organic” by the USDA; (2) A surface organic soil layer (O-horizon, containing 20% or more organic carbon) exceeding four inches in thickness; (3) Subsoils within two feet below grade exhibiting organic carbon concentrations exceeding 5%. In addition to the organic matter in the soil, organic matter that comes in contact with halide-salts, such as decaying plant or leaf litter, which is above the soil or on bare payment is also a condition that may be prone to organohalide formation. HVOCs from this source can also affect the soil and groundwater. Hence, observations of organic matter located above or near the soil is also contemplated as a threshold condition.

Soil pH—The acidity of soil at a given location is considered to exceed its screening threshold when its pH is below about 4.5 Standard Units. Such soils are described as “Extremely Acidic” in USDA soil survey reports.

Iron Content of Soil—The iron content of soil at a given location is considered to exceed its screening threshold when any of the following conditions are present: (1) Iron-cemented horizons and/or iron concretions; (2) Abundant iron as mineral coatings on soil grains or as components in the mineral makeup of the soil (e.g., hematite, limonite, goethite and glauconite); (3) Areas with elevated iron concentrations resulting from geologic conditions (e.g., bog iron deposits and other iron-rich rocks and unconsolidated sediments); (4) Areas where industrial and other activities of man may cause locally elevated iron concentrations (e.g., landfill leachate, mining areas).

Moisture Content of Soil—The moisture content of soil at a given location is considered to exceed its screening threshold when either of the following conditions are present: (1) Available water capacity, as described in USDA soil survey reports, exceeds 3.2 inches in a 60-inch soil profile (verbally described as “moderate” or “high”) or exceeds; (2) Other qualitative descriptions exist which indicate a soil type which tends to remain moist, rather than easily becoming dry or waterlogged.

Thresholds values or conditions should be considered as “occurring together” when they occur: (1) Within the same USDA soil series or other similarly described soil unit; (2) Within two adjacent USDA soil series or other similarly described soil units; or (3) Otherwise in proximity to one another, such that significant chemical, physical or biological interaction may take place.

After data for these factors have been obtained and screened as described, maps can be prepared which show areas exceeding screening thresholds. Viewed together on an overlay map, areas where multiple threshold conditions occur together can be readily identified, providing an indication of areas with the greatest probability for occurrence of salt-enhanced in-situ halogenation of organic matter.

Maps can be prepared manually (hand-drawn) or electronically, using computer aided design (CAD) or geographic information system (GIS) software applications. Increasingly, spatial data such as those collected in the screening study are available in electronic format. In addition, government agencies responsible for highway maintenance usually have access to such software and some make extensive use of GIS. Therefore, use of CAD and GIS may be a cost-effective approach for map preparation and data analysis, particularly for larger screening studies along roadways.

Step 2: Field Investigation

Up to three consecutive stages of activity can be undertaken for the field investigation, including an initial field inspection, a site investigation to identify the presence or absence of HVOC resulting from deicing salt-enhanced in-situ halogenation of organic matter, and a remedial investigation to delineate their source(s) and extent. The field investigation can also facilitate the site-specific application of the remedy. An additional objective pursued during each stage of the field investigation involves distinguishing the presence of HVOCs from those associated with other sources, such as releases from sanitary sewer systems, leaking water supply mains and chemical spills at contaminated sites. In practicing the present invention, not all stages are required and the stages can be eliminated altogether.

However, if undertaken, such a phased approach to the field investigation is consistent with, and based upon, current practices and the regulatory framework employed for site remediation in the United States. Similarly, activities for each field investigation phase follow well-established procedures which can be modified easily to provide consistency with requirements of specific regulatory programs. General activities to be considered for each phase of field investigation work are described below.

A. Initial Field Inspection—A field inspection can be performed to evaluate readily observable conditions relevant to deicing salt-enhanced in-situ halogenation of organic matter and to assess whether the field investigation should progress to the site investigation stage. Ideally, this phase should be completed during winter months when deicing operations and many of the related effects can be directly observed. Field observations which could suggest the need to complete a site investigation would include visual or field screening confirmation of conditions identified in the screening study (e.g., visual evidence of abundant organic material and reddish-brown iron oxides in soil, field pH measurements). The area adjacent to the deicing salt applications should be carefully observed and any engineered and natural drainage features noted to allow an assessment of migration pathways for deicing salt and the need for a site investigation. For example, if it is shown that runoff is controlled by a drainage structure and areas adjacent to the deicing salt applications are also paved, salt migration to organic-rich soils at that location would be unlikely and conducting a site investigation would be unnecessary.

In situations where performance of a site investigation is warranted, information should be gathered during the field inspection which will subsequently support distinction between HVOC impacts from deicing salt-enhanced in-situ halogenation of organic matter and those associated with other sources. This involves identification of other possible sources at or near the site, including sanitary sewer and water supply mains and chemical spills at nearby contaminated sites. Potential impacts from adjacent spill sites can be assessed based on observations of land use and through review of regulatory database listings of known contaminated sites. Because sanitary sewer and water supply mains frequently are located beneath roadways, distinguishing salt-related HVOCs from those released from leaks along such subsurface utilities may be difficult. Maps accurately depicting utility locations should be obtained or prepared based upon field measurements during the field inspection. Additionally, owners of the subsurface utilities should be contacted to determine construction details and the age of the lines and whether past releases along the lines have been documented or are suspected.

The field inspection should also identify one or more locations for groundwater sampling during the subsequent site investigation, biased to the extent possible, toward “worst-case” conditions where salt-enhanced in-situ halogenation of organic matter would be expected to be greatest. Appropriate locations are near the downgradient edge of the area of deicing halide-salt application, coinciding with areas where visual observations or field screening indicate greatest evidence of threshold condition that may have been identified in the screening study. Groundwater flow direction may be determined as known in the art, and based upon a combination of sources, including piezometric data from existing wells, reports for nearby sites and interpretation of surface topography and local drainage features. Care should be taken to select sampling locations where effects of other potential sources of HVOC impacts are minimized. The specific number of sample locations needed is dependant upon site-specific conditions.

B. Site Investigation—During the site investigation, soil evaluation or groundwater sampling should take place in areas identified during the initial field inspection. The objectives of the site investigation are to determine whether HVOCs are present at anticipated “worst-case” locations at concentrations above applicable guidelines (e.g., Federal or State drinking water standards) and to attempt to distinguish between deicing salt-related and other potential sources of such impact. Fall or spring months may provide the best opportunity to observe worst-case effects. During the fall, fungi associated with in-situ halogenation are most active, due to higher soil temperature and moisture content. Abiotic or bacterial in-situ halogenation processes may cause greatest impact to groundwater during the spring, following infiltration of snowmelt and increased precipitation during this period. However, site-specific factors such as low soil permeability and large depth to groundwater may impede such migration and delay the occurrence of maximum groundwater impact. These factors are preferably considered in planning the site investigation.

Although soil sampling can be undertaken to determine the presence of HVOCs in the soil environment, such sampling may be less effective than analyzing nearby groundwater. Most HVOCs partition between the soil solid matrix, pore water, and soil vapor phases, which makes their isolation and precise measurement difficult. Moreover, the formation of HVOCs in the soil may be intermittent based upon certain conditions. However, groundwater act as a reservoir for HVOC formation and accumulation and its analysis is preferred in determining the presence of HVOCs in the soil.

Samples may be collected by a number of methods, including groundwater grab sampling with direct-push (e.g., Geoprobe®) equipment, the use of discrete-interval groundwater samplers (e.g., Hydro-Punch®) and the installation and sampling of temporary or permanent monitoring wells. Sampling of potable wells should not generally be relied upon for site investigation purposes, because such wells are typically not located close enough to deicing areas to evaluate source concentrations. In addition, because potable wells are typically deeper and have longer intake zones than do monitoring wells, such wells may fail to detect shallow groundwater contamination.

Samples from one or more depths within the ten-foot interval immediately below the water table is preferably collected at each of the locations identified during the initial field inspection. Groundwater samples can be analyzed for sodium, calcium, chloride and VOCs (which includes analysis for certain HVOCs). Where sanitary sewers are present near the deicing salt application area, samples should be additionally analyzed for indicators of potential sewage contamination, including ammonia, nitrate, biochemical oxygen demand (BOD) and fecal coliform. Samples can be analyzed by methods acceptable to the regulatory program under which the work is performed. Examples of potentially applicable analytical methods are provided in the Table below. The dates of these methods are as known in the year 2003.

TABLE 1

| Analyte | USEPA Method |
|---|---|
| Sodium | 200.7 |
| Calcium | 200.7 |
| Chloride | 9253, 4500 CLB |
| VOCs (including certain | 624 or 524.2 |
| HVOCs) | |
| Ammonia | 350.1, 350.2 |
| Nitrate | 353.2 |
| Adsorbable Organic | 1650 |
| Halides | |
| Total Organic Carbon | 9060 |
| BOD | 405.1 |
| Fecal coliform | 9222D |

Based on results of the site investigation, a decision can be made regarding the need to proceed with a remedial investigation. If HVOC impacts above applicable groundwater criteria are identified which are not attributable to sources other than deicing salt-enhanced in-situ halogenation of organic matter, the remedial investigation components described below should be implemented to delineate their source(s) and extent. Because significant temporal variation in HVOC formation and transport to groundwater likely occurs, consideration should also be given to performing a remedial investigation even in situations where HVOCs are detected during the site investigation, but at concentrations below applicable groundwater criteria.

Remedial Investigation—During the remedial investigation, additional groundwater sampling is performed to delineate source(s) and the extent of deicing salt-related HVOC impacts to groundwater and to facilitate the site-specific application of a remedy. Sampling of environmental media other than groundwater (e.g., soil and soil gas) can be performed and is included herein, but groundwater sampling is preferred because of the ease of sampling and because the most typical migration pathway for human exposure to HVOC, such as chloroform, is via groundwater. Depending upon site-specific conditions and requirements of the regulatory program under which the field investigation is conducted, performing such sampling may be appropriate during the remedial investigation.

Equipment and procedures for sampling during the remedial investigation may include any of those utilized during the site investigation. The horizontal and vertical extent of HVOC impact to groundwater should be delineated in sufficient detail to facilitate remedy implementation. Typically, this involves collection and analysis of a fairly large number of samples. Following a conventional approach with sample analysis by fixed-base laboratories, several phases of sampling may be required to complete delineation. Therefore, consideration should be given to the use of field screening techniques and/or certified mobile laboratories as a means of expediting and minimizing costs associated with the remedial investigation.

One aspect in the determination of the remedial investigation is to determine an area of elevated HVOC groundwater impacts (i.e., significantly exceeding applicable groundwater criteria) near the edge of the deicing salt application area. This area and the area from which deicing salt entering soil of this area originates can be the focus of remedial activities. Delineation groundwater sampling will identify the areas of elevated groundwater impact. To assess migration pathways for salt causing such impacts, additional activities which can be performed during the remedial investigation include inspection and mapping of surface topography, drainage features and runoff conditions; and reviewing the effects of snow removal by plowing, splashing by traffic and any other relevant mechanisms on distribution of snow and salt on soil adjacent to the deicing salt application area.

Permanent monitoring wells can be installed and surveyed for elevation and horizontal position by a licensed land surveyor during the remedial investigation. The number and location of wells installed is preferably sufficient to allow site-specific determination of groundwater flow direction and to provide for groundwater quality monitoring during subsequent remedial action. Because the objective of the remedy described herein is to remediate deicing salt-related HVOC sources, monitoring wells for remedial action need only be installed in source areas identified during the site investigation and remedial investigation. Wells at other locations (e.g., plume and downgradient “sentinel” wells) may be required at other locations as part of a remedy which may be developed to address an overall contaminant plume.

As noted for the site investigation phase, temporally varying conditions such as salt application, soil biological activity and infiltration may significantly affect remedial investigation results. Therefore, it is preferred that these factors be considered in planning the remedial investigation. In certain instances, quarterly monitoring of groundwater quality can be conducted over a one-year period during the remedial investigation, to assess temporal variations in groundwater impact. Data from such monitoring would provide a basis for deciding upon the need to perform delineation and selecting an appropriate season for doing so at a time of maximum anticipated groundwater impact.

During each remedial investigation groundwater sampling event, field determinations of depth to groundwater can be made at all monitoring well locations as, for example, by using an electronic water level indicator. Groundwater samples can be analyzed for sodium, calcium, chloride and VOCs by the same analytical methods used during the site investigation. During each event, groundwater flow direction can be evaluated during each event by construction of groundwater elevation contour maps.

Once an analysis of the area has been undertaken to determine a threshold condition for halide-salt enhanced halogenation of organic matter in the soil, the present invention contemplates reducing the formation of any potential HVOC in the soil. In one aspect of practicing the present invention, the severity of a threshold condition is reduced to prevent the formation of at least one HVOC in the soil. It is contemplated when the severity of the threshold conditions is reduced, soil that is to be exposed or was exposed to at least one halide salt which was applied for deicing at or near the soil will reduce the formation of HVOC in the soil and consequently reduce any contamination of groundwater by HVOC formed in the soil.

As described above, one aspect of practicing the present invention involves defining an area having a threshold condition for the formation of HVOC. Another aspect of the present invention involves implementing procedures to reduce the formation of HVOC in the identified area.

Implementing remedies to address sources of HVOC groundwater contamination resulting from deicing halide-salts can include four basic components: Definition of Remediation Areas; Primary and Residual Source Treatment; Supplemental Source Treatment; and Groundwater Monitoring.

Details regarding each of these components are provided below. Advantages of this approach include the means of determining salt contribution areas causing HVOC groundwater impact and general remedial design considerations.

As a first step in the remediation process, a potential remediation area is defined having a threshold condition. The actual need for remediation can be determined through site-specific investigations, which will also identify primary and residual source areas contributing to HVOC groundwater. The primary source area is defined as the area from which applied deicing salt may migrate to adjacent soils resulting in the formation of a residual source in the soil where salt enhancement of in-situ halogenation of organic matter takes place. The primary source area may be actual (i.e., in instances where salt application has already caused an impact) or hypothetical (e.g., where salt has not yet been applied, as in the case of a newly constructed road). Residual source areas are determined based upon where the area of roughly the highest HVOC in groundwater are located, which are typically near the edge of the deicing salt application area.

After the remediation areas have been identified, a remedial design is prepared which can include some combination of treatment and monitoring. Details regarding the nature and scope of activities which should be included in the design are discussed below for each of the remedial components. Depending upon the requirements of the regulatory program under which the remediation is performed, work plans, reports and other project documentation may need to be prepared.

Of the factors identified which may contribute to deicing salt-enhanced in-situ halogenation of organic matter and resulting groundwater contamination by HVOCs, deicing-related chloride influx and soil pH are the two which can be most readily manipulated as part of a source remediation program. In practicing one aspect of the present invention, remediation of contaminated soil involves reducing the influx of halide-salts, i.e., chloride salts, from the deicing area (primary source treatment) and to diminish or eliminate in-situ halogenation of organic matter in adjacent soil areas impacted by migration of previously applied salt (residual source treatment).

In the United States, a “bare pavement” policy is followed for roadway snow and ice removal, so simply eliminating deicing chemical use is not a viable option. At the same time, any practical reduction in the amount of salt applied to roadways would be unlikely to decrease chloride migration to the adjacent soil to an extent sufficient to eliminate salt enhancement of in-situ halogenation processes. This also applies to other areas where deicing takes place, such as parking lots, sidewalks and other paved areas, and airport runways. Therefore, the remediation of primary sources of HVOC impacts to groundwater resulting from deicing salt application preferably includes the continuation of deicing operations while preventing new influxes of halides to the roadside environment.

In one embodiment of practicing the present invention, HVOC formation can be reduced by reducing the influx of halide-salts at or near the soil. This can include substituting at least a portion of at least one halide-salt, which is applied for deicing, with a nonhalide-salt to reduce the influx of halide-salts to the soil. Application of an appropriate acetate- or formate-based chemical or agriculturally derived product within a primary source area in lieu of halide-salt applications during deicing operations can be implemented. Nonhalide-salts that can be used for in the present invention include those that known for deicing. Examples of manufacturers are as provided in Table 2 below.

TABLE 2

| | | Liquid/ | |
|---|---|---|---|
| Chemical | Product Name(s) | Solid | Manufacturer |
| Calcium magnesium | CMA ® | Solid | Cryotech |
| acetate | | | |
| Sodium acetate | NAAC ®, Clearway 6s | Solid | Cryotech, |
| | | | Jarchem |
| Potassium acetate | CF7 ®, Clearway 1 | Liquid | Cryotech, |
| | | | Jarchem |
| Sodium formate | Peak SF ®, Safeway | Solid | Old World |
| | SF | | Ind., Clariant |
| Potassium formate | Aviform L50 | Liquid | HydroAgri |
| Corn-, or other | NC-2000, NC-3000 | Liquid | Glacial |
| agriculturally-based | | | Technologies |
| product | | | |

Each of these chemicals are known for use in deicing and are sometimes used as alternatives to chloride salts, but it is believed that none have been employed for the purpose of remediating sources of deicing salt-related HVOC impacts to groundwater. Because the reasons for their proposed application will include continuation of deicing, manufacturers' directions for use of the products is preferably followed. In general, solid products can be applied using the same equipment required for salt application. Liquid products will require special spraying equipment.

Because none of the forgoing products are formulated with halides, such as chlorine or bromine, their use in place of a halide-salt will reduce or eliminate halide migration from primary source areas. In addition, the acetate- and formate-based products produce alkaline solutions in water, so their inevitable transport by runoff, plowing and splashing will likely lead to an increase in the pH of soils adjacent to the deicing area. The increase in pH is expected to reduce or eliminate in-situ halogenation processes, allowing halides, such as chloride, remaining from past salt applications to be naturally flushed from the roadside environment without contributing to formation of HVOCs. Therefore, this remediation technique for addressing primary sources will consequently result in remediation of residual sources. Additional measures may be taken to achieve additional or more rapid remediation of residual source areas, as appropriate, or as an alternative to the use of non-halide salts for deicing.

The products listed above were developed initially for use as less damaging deicing alternatives to chloride salts. The products, particularly the acetate-based ones, have undergone extensive evaluation and been found to cause minimal environmental impairment relative to those associated with chloride-containing products. Because conditions at individual sites may vary significantly, the possibility of adverse side effects should preferably be considered and appropriate regulatory approval obtained prior to their use in some instances.

Treatment of Residual Sources

Although substituting non-halide salts in deicing chemicals is expected to result in significant reduction in the enhancement of in-situ halogenation of organic matter and HVOC formation by residual chloride within the soil, site-specific conditions may warrant performing supplemental or alternate actions to address such residual sources. Supplemental treatment might be employed where more rapid- or more complete-remediation of impacts from residual sources is desired than can be achieved employing only reducing the influx of halide-salts. Also, factors such as product cost and scope of the required application may preclude a complete exchange of the halide-salts for deicing. In such cases, additional or concomitant remedies could be implemented as a measure to minimize enhancement of in-situ halogenation of organic matter and HVOC groundwater impacts resulting from continued salt use.

In another embodiment in practicing the present invention, fertilizer and/or soil amendments such as lime, can be applied to the soil in the residual source area. Studies have shown that use of a nitrogen/phosphorus/potassium (NPK) fertilizer blend is associated with diminished in-situ halogenation of organic matter. Studies provide evidence of a relationship between acidic soil conditions (which can be made more neutral by addition of lime) and increased in-situ halogenation of organic matter. NPK fertilizer application and lime amendment of soils are therefore proposed as options for supplemental or alternate residual source treatment.

Lime soil amendment and NPK fertilizer have a long record of use in agriculture and landscaping, but neither has been employed for the purpose of remediating sources of deicing salt-related HVOC impacts to groundwater. Because the goals of the proposed applications for remedial purposes are consistent with those for use of the products in agriculture and landscaping (i.e., improving soil structure and nutrient content), suppliers' directions for use of the products can be followed.

Lime amendment and fertilizer application are most efficient when the materials are worked into the soil through plowing, discing or other mechanical method and such practices should be followed whenever practicable. Where existing landscaping or other features in the area to be treated preclude mechanical incorporation, the material may be applied as a topdressing on the land surface. For established turf, the lime and fertilizer are best applied following core aeration of the grass-covered area.

The term “lime” refers to a number of amendments used to raise soil pH, including pulverized limestone (calcium carbonate) and dolomitic limestone (calcium/magnesium carbonate), burnt lime (calcium hydroxide) and hydrated lime (calcium hydroxide). The type of lime used depends upon site-specific conditions such as vegetation type and can be determined on a case-by-case basis. The amount of lime required to raise the soil pH by a given number of units depends upon the type and purity of lime used, the fineness of its grind, the depth of incorporation within the soil and the reserve soil acidity. Reserve soil acidity should be determined for soils in the residual source area by submitting a representative number of samples to an agronomic testing laboratory for analysis and determination of the SMP lime test index, or buffer pH. Based on results of this testing, the lime type, purity fineness of grind and application method, the material application rate (e.g., number of pounds per thousand square feet) required to raise the soil to a given target pH can be determined by the laboratory.

Lime amendment and fertilizer application can be performed in a manner consistent with maintenance of existing vegetation in the area. For example, based on nutrient availability, a target pH of between 6 and 7 for mineral soils and between 5.4 and 6.2 for organic soils may be appropriate in many circumstances. However, where acid-loving plants such as rhododendrons, azaleas and certain evergreens are present, it may be preferable to maintain more acidic soil conditions.

Used properly, the lime and NPK fertilizer proposed for use are often regarded as beneficial in providing improved soil structure and nutrient content. Misuse of the products may cause environmental impairment (e.g., excess application causing fertilizer runoff leading to eutrophication of surface water bodies). Because conditions at individual sites may vary significantly, the possibility of adverse side-effects must be considered thoroughly and appropriate regulatory approval obtained prior to lime and NPK fertilizer use.

In many cases, a groundwater monitoring program will be helpful to document the effectiveness of the remediation. The frequency of such monitoring will vary from site to site, depending upon conditions identified during the field investigations and any requirements of the regulatory program under which remediation may take place. For many sites, however, sampling of key source area monitoring wells on a quarterly basis will be sufficient to document the effectiveness of the remediation.

During each groundwater monitoring event, field determinations of depth to groundwater should be made at all monitoring well locations using an electronic water level indicator. Groundwater samples collected from source area wells should be analyzed for sodium, calcium, chloride and VOCs by the same analytical methods used during the site investigation and remedial investigation. As during the remedial investigation, groundwater flow directions should be evaluated during each event by construction of groundwater elevation contour maps.

The duration of activities described above may vary significantly from site to site, depending upon conditions identified during the field investigations, the manner in which remedial action is implemented and requirements of the regulatory program under which remediation takes place. However, it is expected that significant HVOC concentration reductions will in many cases be realized within one or two years after implementation reduced halide flux to the soil.

Following achievement of remedial objectives, actions should be taken to prevent recurrence of salt-enhanced in-situ halogenation of organic matter and HVOC groundwater impacts. Use of a non-chloride containing deicing product should be continued in instances where this has been implemented. Lime and/or NPK fertilizer applications should be continued until a non-chloride containing deicing product can be substituted for previous applied halide-salt.

### EXAMPLES

### 1. Field and Analytical Methodologies

Sample analyses were performed in accordance with SW846, Test Methods for Evaluating Solid Waste, Physical/Chemical Methods, 3rd Edition, 3rd Revision, January 1995. Laboratory data was generated during the project by NJDEP-certified laboratories. Based on review of the data, there were no analytical inconsistencies which required action to be taken on the associated data. Overall, the data quality of the samples was excellent and sufficient to support the objectives of the field studies performed.

1.1 Hydraulic Monitoring and Confirmation of Elevated Chloroform Concentration at the Site of Interest

During each water-level monitoring event, water levels were measured at the site of interest and other wells and staff gages of the site monitoring network. Measurements of both groundwater and surface water levels were made using an electronic water-level indicator. At groundwater monitoring well locations, measurements of depth-to-groundwater were made from the surveyed reference point on the top of the PVC well casing. Depth-to-surface water measurements were recorded at staff gage locations, referencing the surveyed top of the metal rebar at G-1 and the surveyed invert of the corrugated metal pond inflow pipe at G-2. The depth-to-groundwater and depth-to-surface water measurements were subtracted from known elevations of the measurement reference points, to determine groundwater and surface water elevations for each location.

1.2 Groundwater and Soil Sampling

During the investigation, direct-push drilling equipment was used to advance borings for collection of groundwater and soil samples. Groundwater grab samples were collected either from a screen-point sampling device or temporary PVC well set to a specific depth in the borehole. The screen-point sampler consisted of a decontaminated, stainless-steel screen, with a retractable stainless-steel sleeve to protect the screen during driving the device to the desired sampling depth. Temporary PVC wells consisted of new, one half-inch diameter PVC well screen and solid riser material. In general, the screen-point sampler was used to collect samples from below the water table, while temporary PVC wells were set with screens straddling the water table to collect shallower samples. Groundwater samples were withdrawn from the screen-point samplers and temporary PVC wells using new ⅜-inch outside diameter, polyethylene tubing and an attached stainless-steel check valve. Sample collection was accomplished by slowly raising and lowering the tubing and check valve assembly within the well, filling the tubing. Subsurface deposits in the sampling area exhibited relatively low permeability and minimal recharge capacity. Therefore, to prevent loss of VOCs due to drawdown and resulting sample aeration, no further purging was conducted. Samples were collected by removing the tubing from the well and transferring the contained water to laboratory supplied glassware.

Soil cores were collected for visual inspection and collection of samples for laboratory analysis at one of the borings (designated S-1), located immediately upgradient of the site of interest and at the downgradient edge of a concrete walkway leading to an upper parking lot. Each core was collected directly into a new acetate core liner, four feet in length. For each soil core, information recorded included depth intervals, percent recovery and a soil description including color, grain size, moisture content and any evidence of contamination (e.g., stain or discoloration, odors, PID readings). Samples were collected from selected six-inch intervals for laboratory analysis of VOCs, following NJDEP's specified methanol preservation sampling procedure.

1.3 Analytical Methods

All samples collected during the investigation were analyzed by NJDEP-certificatied laboratories. Samples were analyzed for VOCs, by USEPA Method 8260B, modified to achieve detection limits below cleanup criteria for all analytes. Sample results were reported according to the format specified for New Jersey Level IV QC & Data Packages-Reduced Laboratory Data Deliverables for Non-USEPA CLP Methods.

1.4 Quality Assurance/Quality Control (QA/QC)

In order to ensure the quality, reproducibility and completeness of data collected during sampling activities, certain QA/QC procedures were implemented during field activities. These procedures are discussed below.

1.4.1 Equipment Decontamination

To reduce the possibility of cross-contamination, any equipment that may have come in contact with soils or groundwater was properly decontaminated utilizing the following procedure. All utensils and downhole equipment (direct-push drill rods and soil coring equipment, and stainless steel bowls and trowels) were decontaminated prior to use according to the following steps:
1. Potable water rinse
2. Wash in non-phosphate detergent solution (e.g., Liquinox)
3. Deionized water rinse
4. 10% nitric acid rinse
5. Deionized water rinse
6. Methanol rinse
7. Deionized water rinse
   Where possible, disposable items were utilized, to reduce the potential for sample cross-contamination.

1.4.2. Sample Delivery and Custody

Immediately upon collection, all samples were individually labeled, sealed and packed on ice in a cooler in the laboratory. During each day of sampling, a chain-of-custody record was maintained for the samples collected that day. Transfer of samples to the laboratory took place under proper chain-of-custody procedures.

### 2. Remedial Investigation Results

2.1 Hydraulic Monitoring and Confirmation of Elevated Chloroform Concentration at the Contaminated Site

It was determined at the outset of the investigation that aquifer recharge following precipitation and/or snowmelt events resulted in intermittent influxes of chloroform to groundwater, resulting in the varied concentrations observed at the contaminated site during the groundwater investigation. Because of the intermittent nature of the release, it was considered necessary to perform source delineation during a period when the source was suspected to be active. Hydraulic monitoring was performed as an indirect means of estimating when such conditions were present. Following observation of a significant increase in groundwater elevation at the site, groundwater grab samples were collected from the contaminated site to confirm the presence of elevated VOC concentrations. Analytical results of the screening indicated chloroform concentrations of 87 to 89 ug/L, which were close to the maximum concentration of 97 ug/L, detected at the contaminated site during the previous groundwater investigation.

2.2 Groundwater Grab Sampling

Following the hydraulic monitoring and confirmation of elevated chloroform concentrations, a thorough and detailed delineation of chloroform concentrations in groundwater near the site was accomplished during the investigation by collection and onsite analysis of groundwater grab samples. Samples were collected from direct-push borings located along five separate transects, oriented approximately perpendicular to the direction of groundwater flow. Samples were first collected along a transect passing through the contaminated site location, extending along the north-south trending facility driveway. To evaluate concentrations upgradient of the site, two transects were then completed in alignment with the western and eastern edges of the upper parking lot. To complete the downgradient delineation, samples were collected along an additional transect located downgradient of the site. One final transect was then completed along the north-south centerline of the upper parking lot, to rule out the possibility of a source beneath this area.

Samples were generally collected first from borings along the center of each transect, and subsequently from other locations along each transect as necessary to find the “edge” of the plume (i.e., below GWQS chloroform concentration). Samples were collected and analyzed at all locations on the three transects upgradient of the contaminated site, despite findings of low (below GWQS) or non-detect results for chloroform at central locations. This was done because groundwater flow direction in this area was inferred based on limited piezometric data. Under such conditions, groundwater flowpaths cannot always be reliably estimated for purposes of tracing contaminant detections to upgradient sources, and additional sampling and analysis was warranted to define upgradient conditions.

The groundwater grab sampling program identified an area of groundwater exhibiting above-GWQS chloroform concentrations, extending in a westerly and southwesterly direction from the western edge of the upper parking lot to a point approximately 100 feet downgradient of the contaminated site. The highest detected chloroform concentration (110 ug/L) was noted for a grab sample collected from the contaminated site during the delineation program. Sample analyses performed to vertically delineate the chloroform detections indicate that above-GWQS chloroform concentrations (and at most locations, chloroform detections) are restricted to the upper five feet or so of the saturated zone. Attempts to collect deeper samples were impeded and at some locations unsuccessful, due to the presence of unweathered, low-permeability clayey silt at depth.

Based on the horizontal distribution of measured chloroform concentrations, the central portion of the chloroform plume passes through the contaminated site, arcing in a westerly and southwesterly direction toward a monitoring well, located along the southern property boundary. This configuration is consistent with that which would be predicted based on piezometric data, because interpreted groundwater flowpaths in this area of the site follow a similar trend. In addition, previous groundwater monitoring results for wells along the southern property boundary support the conclusion that the plume is directed toward the monitoring well.

The overall plume configuration and distribution of elevated chloroform detections indicate that none of the hypothetical sources previously identified (i.e., past discharges to building drains and storm sewers, sanitary sewers) can account for the chloroform impact to groundwater. Were residual impacts from past discharges to building drains and storm sewers the cause of the chloroform impact, the origin of the plume would be located downgradient of the contaminated site, near the storm sewer lines and measurable concentrations of chloroform might be expected in the storm water currently in use. Similarly, elevated concentrations in groundwater adjacent to and downgradient of the sanitary sewer lines would be expected if an unidentified leak along the lines were the source of the chloroform impact to groundwater. None of these conditions was observed during the investigation.

As the chloroform delineation program progressed, it became evident that none of the hypothetical sources previously identified (i.e., past discharges to building drains and storm sewers, sanitary sewers) could account for the observed chloroform distribution in groundwater. Similarly, the possibility of other contributing sources was ruled out by the delineation sampling results.

2.3 Source Evaluation

Based on the findings, it was believed that chloroform detected in groundwater near the site originates in the shallow soil zone as a result of in-situ biological chlorination of humic substances in surface soil and decomposing leaf litter in this area. Research and site conditions further suggested that in-situ chlorination may be augmented by influxes of chloride in runoff, as a result of salt application for ice removal on the adjacent roadway and sidewalk areas.

The detailed delineation program performed during the investigation indicates that chloroform impacts originate in the area between the site and the upper parking lot. This area is traversed by a concrete walkway and stairs which lead to the upper parking lot, but is otherwise undisturbed by construction or other site operations. The ground surface is grass-covered and the mature tree cover in the area results in periodic accumulation of abundant leaf litter throughout the area and along the margins of the upper parking lot, located immediately up-slope. An organic topsoil layer was noted during completion of soil borings in this area. Based on soil survey maps (USDA 1989), shallow soil near the area of interest consists of sandy loams of the Collington Series, which are classified as strongly- to extremely-acidic (pH of about 3.6 to 5.5).

The owners employ best management practices for roadway, parking area and sidewalk deicing, including use of a 50/50 mixture of ASTM-D-632-84 deicing salt and sand for roadways and parking areas, and use of potassium chloride salt for sidewalks. To minimize potential impacts to storm water, these materials are used in moderation, only as required for safety. Nonetheless, it is inevitable that melt-waters resulting from the chloride salt application and precipitation runoff from the treated areas periodically contain significant concentrations of chloride. During investigation field activities, precipitation runoff from the upper parking lot was observed to flow over the concrete stairs and walkway at the western edge of the parking lot and onto a soil covered area immediately upgradient of the contaminated site.

Conditions in the area between the site and the upper parking lot appear optimal for the occurrence and road salt-related enhancement of in-situ biological chlorination processes. Specifically, humic materials are present in abundance in the upper soil horizon and in the form of decaying leaf litter along the edges of the parking lot and driveways. The shallow soil exhibits acidic conditions, as the decaying leaf litter would also be expected to do, through the release of tannic acid. Runoff from the upper parking lot and concrete stairs and walkway introduces an abundant pool of chloride, which likely enhances the rate of in-situ chlorination in the soil and grass covered areas. Decaying leaf litter along the edges of the parking lot and driveways is at least periodically in close or direct contact with salt applied to the pavement in these areas. Salt-enhanced biological chlorination of humic material may therefore also take place in the decaying leaf litter, resulting in runoff containing chloroform.

Consideration of site conditions also provide an understanding of mechanisms by which such salt-enhanced in-situ biological chlorination might result in the observed chloroform impact to groundwater. Specific processes identified in the literature include downward flushing from the shallow soil zone with infiltration of precipitation and/or snow-melt and vertical migration in soil gas, either through diffusion or due to vapor density. Such conditions would explain the occurrence of chloroform in groundwater beneath soil and grass covered areas adjacent to the upper parking lot and concrete stairs and walkway.

Direct infiltration of runoff containing chloroform formed in leaf litter is another mechanism which may contribute to the observed distribution of chloroform in groundwater. The observed below-GWQS chloroform detections at several locations beneath the northern portion of the parking lot and along the driveway north of the site and the concrete walkway may be explained by such a scenario. However, the presence of chloroform in these areas could also result from lateral migration in the vadose zone, either in the soil gas or pore water phases, prior to reaching the water table, or from in-situ halogenation processes as described, because piling of snow plowed from the parking lot in this area may release salt to the soil.

Results of groundwater monitoring at the site to date are consistent with the above explanation of chloroform formation in the shallow soil. Because microbial activity in the shallow soil zone is diminished during the cold and dry winter and dry summer months, the rate of in-situ chlorination is also expected to be low at those times. Chloride transported to the soil area upgradient of the site during winter and early spring would remain in the shallow soil until flushed away by infiltration, contributing to in-situ chlorination processes during warmer, wetter periods of greater biological activity (later spring and autumn).

The appearance of chloroform in groundwater would be expected to lag its formation in the shallow soil zone. Because transport from the shallow soil zone to groundwater is largely dependent upon infiltration, sustained periods of precipitation and/or snowmelt might be necessary to cause a significant influx of chloroform to groundwater. Therefore, it is possible that chloroform created during an autumn period of microbial activity might reside in the vadose zone during a dry- or cold weather period (possibly as soil gas beneath frozen surface soils), before being flushed downward to the water table. Such conditions may account for the detection of elevated chloroform concentrations at the contaminated site during the early spring investigation field activities.

### 3. Delineated Extent of Chloroform in Groundwater

Based on the relationship between groundwater elevation and chloroform concentration at the site, a hydraulic monitoring and confirmatory sampling were performed as preliminary tasks during the investigation. These activities help to identify an appropriate time to initiate the delineation sampling program, ensuring that delineation encompassed the full extent of chloroform groundwater impacts and maximizing the possibility of tracing the detections to a source.

3.1 Source of Chloroform

The source of chloroform impacts to groundwater was evaluated based upon detailed mapping of the extent of chloroform in groundwater, soil sampling immediately upgradient of the area with highest groundwater concentrations of chloroform and through a review of literature pertaining to natural mechanisms of chloroform formation in soil and subsequent transport to groundwater. Chloroform concentrations in groundwater were found to originate in a relatively undisturbed grass-covered area between the site and the upper parking lot, away from any known or likely past site activities which could have resulted in a chloroform discharge. Field screening with a PID and soil sample analysis indicated no evidence of soil impacts in this area.

Based on this investigation, it was concluded that chloroform detected in groundwater near the site originated due to a combination of natural soil processes and site-related activities. Specifically, chloroform is believed to form naturally in the shallow soil zone as a result of in-situ biological chlorination of humic substances in surface soil and decomposing leaf litter. This process is believed to be augmented by influxes of chloride in runoff, as a result of chloride salt application for ice removal on the adjacent roadway and sidewalk areas. The processes which cause migration of chloroform to groundwater include, downward flushing with infiltration from the shallow soil zone and vertical movement in the in soil gas phase, either through diffusion or due to vapor density.

### 4. Remediation

Notwithstanding implementing best management practices for roadway, parking area and sidewalk deicing, there were indications that chlorides associated with ice removal may have the unintended consequence of enhancing natural, in-situ chlorination processes, resulting in above-GWQS concentrations of chloroform groundwater. Therefore, reducing the contact and/or chemical/biological interaction of chlorides resulting from salt application for ice removal with humic materials in adjacent soil areas and decaying leaf litter was necessary.

Reduction of the HVOCs can be achieved by carrying out one or more of the following steps: (1) further minimizing salt usage in ice removal, including switching to use of a sand/salt mixture along the concrete stairway leading to the upper parking lot, to minimize chloride runoff to adjacent soils; (2) modification of stormwater drainage and plowing practices to prevent direct runoff from the upper parking lot to the area upgradient of the site; (3) inspection and repair of any cracks in the parking lot pavement in the area upgradient of the site; (4) more-frequent removal of leaf litter from areas at the edges of the upper parking lot and ensuring that salt application for ice removal avoids any such areas which may periodically accumulate; and (5) landscape application of a lime and NPK fertilizer mixture to surface soil in the area between the site and the upper parking lot, to promote a more neutral soil pH and conditions less-favorable to the in-situ biological chlorination of humic materials in soil.

In addition to these remedies, localized use of calcium-magnesium acetate (CMA) or another similar, non-halide containing deicing product on the concrete walkway leading to the upper parking lot should be used. Though the cost of such alternative products (as much as twenty to forty times the cost of traditional, chloride-containing deicing products) precludes their widespread use, such localized use would likely result in a significant reduction in transport of chloride to the soil. Additionally, CMA is manufactured from dolomitic limestone and its alkaline pH offers a possible secondary benefit, because its runoff would tend to neutralize the adjacent naturally acidic soils (National Research Council. 1991). This in turn may diminish the effects of any existing chloride residues resulting from past salt applications, by creating conditions unfavorable to in-situ biological chlorination processes.

By implementing one or more of these remedies, and preferably all of the remedies, the influx of halide, particularly chloride, is expected to be terminated at its source and a significant reduction in HVOCs in the surrounding soil should be achievable within a year. It is expected that the implementation of these remedies would also achieve an approximate 75% reduction in the formation of HVOCs within two years and the maintenance of groundwater with chloroform contaminates of HVOC within government guidelines, as for example, groundwater that does not contain more than about 6 ug/L of chloroform due to deicing activities.

In this disclosure there is shown and described only the preferred embodiments of the invention and but a few examples of its versatility. It is to be understood that the invention is capable of use in various other combinations and environments and is capable of changes or modifications within the scope of the inventive concept as expressed herein.

1. A method of preventing at least one halogenated volatile organic compound (HVOC) in soil, the method comprising:
evaluating soil that is exposed to deicing activity to determine a threshold condition for halide-salt enhanced halogenation of organic matter in soil; and
reducing the severity of the threshold condition of the soil that is to be exposed or was exposed to at least one halide-salt applied for deicing at or near the soil to prevent the formation of the at least one HVOC in the soil.

2. The method of claim 1 wherein the threshold condition is selected from the group of soil conditions consisting of: organic matter content, iron content, soil acidity, halide input, moisture content, HVOC content and combinations thereof.

3. The method of claim 1 comprising evaluating soil by carrying out a screening analysis.

4. The method of claim 1 wherein the soil contains at least one HVOC and further comprising determining whether the at least one HVOC is a result of one or more halide salts.

5. The method of claim 1 wherein the soil contains at least one HVOC that was a result, in part, of halide-salt deicing.

6. The method of claim 1, wherein the threshold condition is soil acidity and comprising increasing the pH of the soil to reduce the severity of the condition.

7. The method of claim 1 comprising changing characteristics of the soil by applying fertilizer to the soil.

8. The method of claim 1 comprising reducing the threshold condition by reducing the influx of halide-salts at or near the soil.

9. The method of claim 8 comprising reducing the influx of halide-salts at or near the soil by applying a calcium magnesium based salt for deicing at or near the soil as a substitute, at least in part, for the at least one halide-salt used for deicing.

10. A method of reducing at least one halogenated volatile organic compound (HVOC) in soil, the method comprising:
evaluating soil, which was exposed to at least one halide-salt that was applied for deicing at or near the soil, to determine the presence of at least one HVOC in the soil; and
reducing the formation of HVOC in the soil.

11. The method of claim 10 comprising reducing the amount of halide-salt used in deicing to reduce the formation of HVOC in the soil.

12. The method of claim 11 comprising substituting at least a portion of the at least one halide-salt, applied for deicing, with a non-halide-salt to reduce the formation of HVOC in the soil.

13. The method of claim 12 wherein the non-halide-salt is calcium magnesium acetate.

14. The method of claim 10 comprising increasing the pH of the soil to reduce the formation of HVOC in the soil.

15. The method of claim 10 further reducing HVOC in groundwater at or near the soil containing HVOC.

16. The method of claim 10 comprising reducing the amount of chloroform in groundwater at or near the soil containing HVOC.

17. The method of claim 10 comprising reducing chloroform in groundwater at or near the soil containing HVOC to about 6 ug/L.

18. The method of claim 10 comprising evaluating soil by analyzing groundwater at or near the soil.
